(19) United States
(12) Patent Application Publication
(10) Pub. No.: US 2026/0325741 A1
(43) Pub. Date: Oct. 22, 2026

(54) DETECTING INFUSION PUMP FAILURE

(71) Applicant: CareFusion 303, Inc., San Diego, CA (US)

(72) Inventor: Michael K. WORKMAN, Carlsbad, CA (US)

(21) Appl. No.: 19/186,508

(22) Filed: Apr. 22, 2025

(51) Int. Cl. A61M 5/142; G16H 40/63

(52) U.S. Cl. CPC A61M 5/142; G16H 40/63; A61M 2205/18; A61M 2205/3553; A61M 2205/50

(57) ABSTRACT

A system and method for detecting failure of an infusion pump is disclosed. The system includes a communication module that is separate and independent of a pump operating system of the pump. The communication module periodically monitors the pump operating system to determine the operational status of the pump operating system, and establishes a failure of the pump operating system when signals are no longer detected from the pump operating system within a specific predetermined timeout interval. The communication module then communicates with a remote computing device regarding a failure to determine the operational status of the pump operating system. According to various examples, the timeout interval is determined based on predetermined technical factors associated with the infusion pump and/or clinical factors associated with a patient receiving a therapy from the infusion pump, the therapy, or a care area associated with the patient.

### BACKGROUND

This application relates generally to ensuring that an infusion device is properly operating.

Modern hospital infusion devices include multiple pump channels for infusing multiple fluids simultaneously. These patient care units (“PCUs”) are mobile and able to administer medications to a patient as the patient moves between care areas throughout a medical environment such a hospital organization. A PCU may incorporate a network interface to connect the PCU to a network so that infusion data can be provided by the PCU to hospital information systems. However, in such systems, the operational circuitry and pump operating systems of infusion devices are tightly coupled to the network interface, presenting clinical challenges. For example, if the operating systems fails, the device will stop communicating altogether and there is no mechanism for providing a remote system any indication of the failure.

### SUMMARY

The subject technology provides a system and method for detecting failure of an infusion device. Unlike legacy systems and processes, the subject technology decouples the main application and/or operating system of the infusion device from the communication systems, and augments a proprietary communication system with monitoring and reporting capabilities. In this regard, the communication system, or communication module, can monitor interactions with the pump operating system and, if no interactions are detected, register an alert, for remote consumption, indicating a failure of the pump's operating system.

According to various implementations, the subject technology provides a method for detecting failure of an infusion pump, comprising: determining a timeout interval for monitoring a pump operating system of an infusion pump based on at least one predetermined technical factor associated with the infusion pump or at least one clinical factor associated with a patient receiving a therapy from the infusion pump, the therapy, or a care area associated with the patient; programming a communication module with the determined timeout interval, the communication module being separate and independent of the pump operating system and configured to communicate with the pump operating system and to communicate with a remote computing device regarding an operational status of the pump operating system; monitoring, by the communication module, the pump operating system at periodic intervals to determine the operational status of the pump operating system; establishing, by the communication module, during the monitoring, that the operational status of the pump operating system cannot be determined within the determined timeout interval; and responsive to the communication module not determining the operational status of the pump operating system within the determined timeout interval, by the communication module, communicating with the remote computing device regarding a failure to determine the operational status of the pump operating system. Other aspects include corresponding devices, systems and computer program products for implementation of the corresponding method and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1A depicts an example of an institutional patient care system of a healthcare organization, according to aspects of the subject technology.

FIG. 1B is another view of a portion of the example patient care unit shown in FIG. 1A, according to aspects of the subject technology.

FIGS. 2A and 2B depict example sequence diagrams of first and second example systems for detecting failure of an infusion device, according to various aspects of the subject technology.

FIGS. 3A and 3B depict example architectural arrangements of the communication module with respect to the pump operating system, according to various aspects of the subject technology.

FIG. 4 depicts a first example process flow diagram for detecting failure of an infusion pump, according to aspects of the subject technology.

FIG. 5 is a conceptual diagram illustrating an example electronic system for detecting failure of an infusion pump, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

Alarm messages generated by infusion systems are typically pushed to consumers in response to a hazardous condition which needs a response. Accordingly, consumers may only expect to receive an alarm responsive to a hazardous condition, creating automation bias. That is, the consumers expect the device to be operational at all times until they receive a message to the contrary. Conventional systems do not account for when the device experiences a failure, so when the device goes down, no messages can be sent to the consumer, and the consumer may believe that the device (or the system in which the device is included) is operating normally.

The subject technology solves the foregoing problem by way of a technical configuration that remains capable of indicating to consumers when an infusion device experiences a total failure or loss of communication. The subject technology incorporates a communication module within the infusion device that is separate and independent of a pump operating system, and which periodically monitors the pump operating system to determine the operational status of the pump operating system. The communication module establishes a failure of the pump operating system when operational activity of the pump operating system cannot be detected within a specific predetermined timeout interval. The communication module may then infer that a failure has occurred and communicates with a remote computing device regarding the failure. As will be described further, the timeout interval is determined based on predetermined technical factors associated with the infusion pump and/or clinical factors associated with a patient receiving a therapy from the infusion pump, the therapy, or a care area associated with the patient.

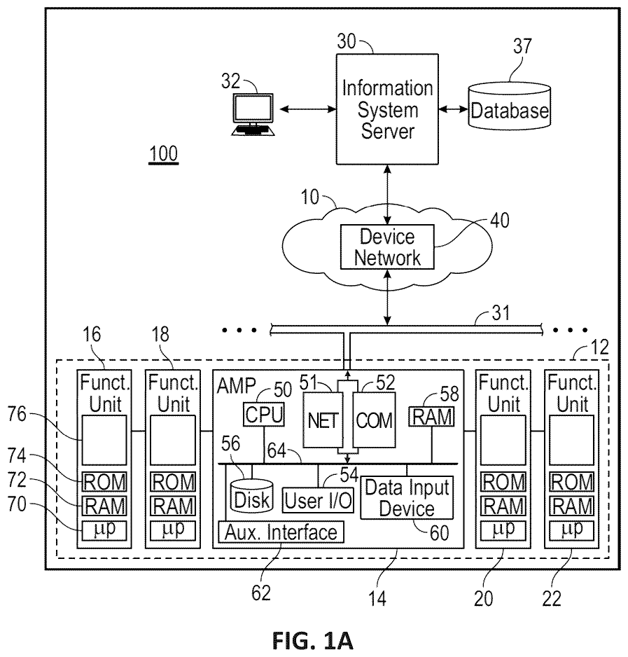

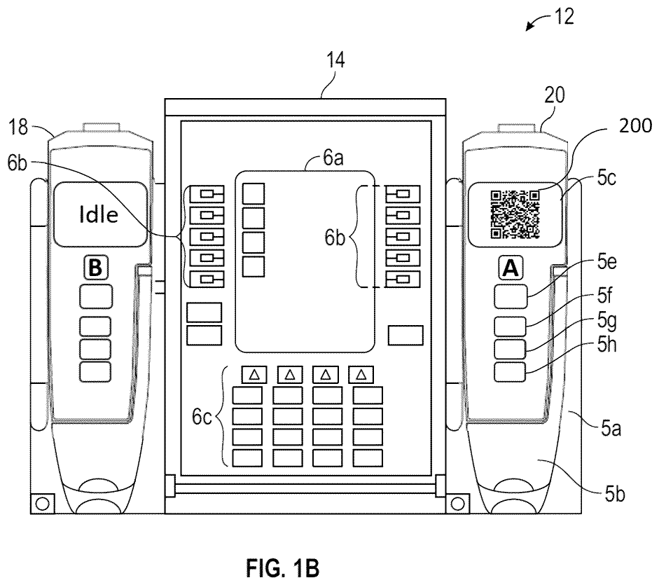

FIG. 1A depicts an example of an institutional patient care system 100 of a healthcare organization, according to aspects of the subject technology. In FIG. 1A, a patient care unit (“PCU” or “medical device” generally) 12 is connected to a hospital network 10. The term patient care unit may include various ancillary medical devices such as an infusion pump, vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each element 12 is connected to an internal healthcare network 10 by a network transmission channel 31. Network transmission channel 31 is any wired or wireless transmission channel, for example an 802.11 standard-based wireless local area network (LAN). In some implementations, network 10 also includes computer systems located in various departments throughout a hospital. For example, network 10 of FIG. 1A optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 41 by which patient care units 12 (and other devices) communicate in accordance with normal operations.

Additionally, institutional patient care system 100 may incorporate a separate information system server 30. Moreover, although the information system server 30 is shown as a separate server, the functions and programming of the information system server 30 may be incorporated into another computer, if such is desired by engineers designing the institution's information system. Institutional patient care system 100 may further include one or multiple device terminals 32 for connecting and communicating with information system server 30. Device terminals 32 may include personal computers, personal data assistances, and mobile devices such as laptops, tablet computers, augmented reality devices, or smartphones, configured with software for communications with information system server 30 via network 10.

Patient care unit 12 comprises a system for providing patient care. Patient care unit 12 may include or incorporate operating system components such as pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care unit 12 comprises a control module 14 connected to one or more functional modules 16, 18, 20, 22. Control unit 14 includes one or more operating systems including, for example, a central processing unit (CPU) 50 connected to a memory, such as a random access memory (RAM) 58, and one or more interface devices such as user interface device 54, a coded data input device 60, and an auxiliary interface 62 for communicating with additional modules or devices. Control unit 14 (and/or its operational systems) may also include a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen (e.g., 6a of FIG. 1B). Additionally, or in the alternative, user interface device 54 could include any means for displaying and inputting information, such as a monitor, a printer, a keyboard, softkeys, a mouse, a track ball and/or a light pen. Data input device 60 may be a code reader capable of scanning and interpreting data printed in a coded format (e.g., a scancode). Additionally or in the alternative, data input device 60 can be any device for entering coded data into a computer, such as a device(s) for reading a magnetic strips, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown as disposed within control unit 14, it is recognized that data input device 60 may be associated with a pharmacy system or located externally and communicating with pharmacy system through an appropriate communication means. Auxiliary interface 62 may be an RS-232 communications interface, however any other means for communicating with a peripheral device such as a printer, patient monitor, infusion pump or other medical device may be used without departing from the subject technology. Additionally, data input device 60 may be a separate functional module, such as modules 16, 18, 20 and 22, and configured to communicate with controller 14, or any other system on the network, using suitable programming and communication protocols.

According to various implementations, the patient care unit 12 further includes a network controller 51 and/or a proprietary communication module 52 that interface(s) with the control circuitry of the PCU and/or pump operating system(s), and for connecting the PCU and its various components to network 10. Controller 51 and/or module 52 include a network interface that may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

In some implementations, network controller 51 is directly connected between the pump operating system and network 10. In some implementations, all communication between the pump operating system and network 10 are handled by the communication module 52. In some implementations, a network controller 51 maintains communications (e.g., network traffic) and the communication module is capable of detecting and/or monitoring such communication. According to various implementations described herein, the communication module 52 is separate and independent of the pump operating system. That is, it may be electronically isolated from the pump operating system(s) such that, if the pump operating systems fail, the communication module 52 maintains independent operation and communication with the network 10 and any remotely connected device/server.

The pump operating system may include, for example, the main application of the pump system—e.g., handling all clinical operations, interaction with the user, and infusions by way of operational control over the pump mechanics. According to various implementations, the pump operating system may include hardware and/or software. In some implementations, a housing of the PCU (or functional module) may include a control circuitry (e.g., CPU 50, memory 58, I/O 54, etc.) configured to execute the pump operating system (e.g., as software) stored at least in part on a memory and to operate the infusion pump according to a set of operating parameters provided to the executed pump operating system. The pump operating system may be configured to provide infusion information pertaining to an infusion to an external server via the network interface 51 and/or communication module 52.

The communication module 52 may include separate, standalone hardware within the physical aspects of the pump mechanics (e.g., within the same housing as a control unit 14 or functional module 16, 18, 20, 22). In this regard, the communication module 52 cannot cause failure of the pump operating system, and vice versa. As described herein, the communication module 52 is configured to communicate with the pump operating systems of the PCU and components thereof, and to communicate with a remote computing device regarding an operational status of the pump operating system(s). In some implementations, the communication module 52 is responsible for routing communications (e.g., infusion data sent by the PCU to remote systems). The primary function of the communication module 52, as described herein, is to monitor the operational status of the PCU and its components, and to report the operational status to a remote system (e.g., server 30), for example, over network 10.

While the communication module 52 is depicted within the control unit 14, monitoring pump operating system(s) within the control unit, the communication module 52 may reside in a functional module 16, 18, 20, 22 or in an external device (e.g., attached or proximate to PCU 12). The communication module 52 may monitor the pump operating system(s) of an individual functional module 16, 18, 20, 22, apart from the pump operating system(s) of the control unit 14, or both simultaneously. In some implementations, an infusion device 12 may include both an integrated control unit 14 together with a pump mechanism within a single housing. In such implementations, the communication module may monitor the pump operating system of the device as either separate hardware within the same housing.

Functional modules 16, 18, 20, 22 are any devices for providing care to a patient or for monitoring patient condition. As shown in FIG. 1B, at least one of functional modules 16, 18, 20, 22 may be an infusion pump module such as an intravenous infusion pump for delivering medication or other fluid to a patient. Each of functional modules 16, 18, 20, 22 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor, an intracranial pressure monitor, or the like. Functional module 16, 18, 20 and/or 22 may also include a printer, scanner, bar code reader, near-field communication reader, RFID reader, or any other peripheral input, output or input/output device.

Each functional module 16, 18, 20 and/or 22 communicates directly or indirectly with control unit 14, with control unit 14 providing overall monitoring and control of device 12. Functional modules 16, 18, 20 and/or 22 may be connected physically and electronically in serial fashion to one or both ends of control unit 14 as shown in FIG. 1B. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care unit 12 through one or more auxiliary interfaces 62.

Each functional module 16, 18, 20, 22 may include module-specific operating systems, including components 76, a microprocessor 70, a volatile memory 72 and a nonvolatile memory 74 for storing information. It should be noted that while four functional modules are shown in FIG. 1A, any number of devices may be connected directly or indirectly to central controller 14. The number and type of functional modules described herein are intended to be illustrative, and in no way limit the scope of the subject technology. Module-specific components 76 include any components necessary for operation of a particular module, such as a pumping mechanism for infusion pump module 16.

While each functional module may be capable of a least some level of independent operation, control unit 14 monitors and controls overall operation of device 12. For example, as will be described in more detail below, control unit 14 provides programming instructions to the functional modules 16, 18, 20, 22 and monitors the status of each module.

Medical devices incorporating aspects of the subject technology may be equipped with a network interface module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care unit 12 and network 10 may communicate via automated interaction, manual interaction or a combination of both automated and manual interaction. Automated interaction may be continuous or intermittent and may occur through direct network connection 52, or through RS232 links, MIB systems, RF links such as BLUETOOTH, IR links, WLANS, digital cable systems, telephone modems or other wired or wireless communication means. Manual interaction between patient care unit 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10. For example, and not by way of limitation, decisions can be made in an information system server 30 (e.g., health information system (HIS) server, decision support, or remote data server), hospital department or unit stations 32, or within patient care unit 12 itself.

All direct communications with medical devices operating on a network in accordance with the subject technology may be performed through server 30. In accordance with aspects of the subject technology, network interface modules incorporated into medical devices such as, for example, infusion pumps or vital signs measurement devices, ignore all network traffic that does not originate from an authenticated remote data server. In some implementations, server 30 may coordinate tracking of the location and status of all networked medical devices that have NIMs, and maintain open communication.

According to various implementations, server 30 may include a formulary and/or pharmacy information system. Pharmacy information systems may enable a safer physician medication order process. A pharmacy website (e.g., provided by the server) may provide the physician with a list of available drugs from which the physician may select (stored, e.g., in a database 37). The pharmacy website may contain a drug library having the list of available drugs but may also contain and present to the physician the drug names associated with recommended dosages and dose limits that have been established or adopted by the healthcare facility. In such a case where the physician need only select items from the computer screen rather than having to manually type in drug names and drug administration numbers (such as infusion rates, times, etc.) associated with administration of the medication, a more accurate medication process should result.

If a clinical order is for administration of a particular medication regimen, the order will be transmitted to the facility's pharmacy information server 30. The pharmacy reviews the order, and once the order has been prepared, the order may be transmitted to the nurse station (e.g., at workstation 32) for matching with the appropriate patient. Formulary is an approved list of drugs for use (e.g., available to order for a patient) within a medical facility. Within a formulary, there may be indication for use information and/or concentrations and drug ranges approved for the facility. As will be described further, a formulary may be used to define one or more medical device drug libraries, which may then be provided to infusion pumps within a hospital network. Inside the library, there is medication information such as drug names, concentration, diluent volume, strength, minimum or maximum infusion parameters for a drug, and other parameters. The formulary's establishment of these parameters, along with parameters for off-formulary orders, via the server 30 is useful for maintaining consistency across the healthcare environment and ensuring an order is intelligible and executed according to expectations by other devices within the system (e.g., an infusion pump).

With further reference to FIG. 1A, patient care unit 12 is capable of operating in several different modes, or personalities, with each personality defined by a configuration database. The configuration database may be a database 56 internal to patient care unit, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care unit's 12 location in the hospital or hospital computer network. Patient care information may be entered through interface device 52, 54, 60 or 62, and may originate from anywhere in network 10, such as, for example, from a pharmacy server, admissions server, laboratory server, and the like.

A memory 56, 58 of the control unit 14 may contain a drug library or libraries, an event log or logs, and pump configuration settings, such as, but not limited to, profiles to be used in particular practice areas such as ICU, PED, etc. The memory may be electronically loadable memory such as non-volatile memory (e.g., EEPROM). Drug libraries stored on pumps (which illustratively contain such information as the drug names, ranges of delivery parameter values such as proper concentrations, dosage units, and dose limits) can be used to perform drug calculation-based infusions in a clinical setting.

A drug library stored within the pump's memory may include clinical order settings such as limits set by the clinical institution for each drug of the library (also termed as “guardrails” herein). Such limits may take the form of maximum and minimum dosages for each drug which may be made dependent on patient factors or other factors associated with delivery of the drug. For example, the dosage limits may vary depending on the weight of the patient or body surface area (“BSA”), depending on the unit or ward of the medical institution in which the drug is being used (for example neonatal care unit (NCU), the intensive care unit (ICU), etc.), and depending on other factors. An alarm may be provided if the nurse sets the pump to operate outside the range between the limits for a particular drug. In some cases, the alarm may be overridden and in other cases it may not. The medical facility may establish “soft” limits for each drug, which may be overridden by the nurse, and “hard” limits which may not. In either case where a limit is exceeded, a pump data log or other processor in communication with the infusion pump may record each such limit event for later analysis where the attempted setting is higher than the maximum or lower than the minimum dosage.

The pump also includes a display for displaying a user interface, including a control panel through which the user can program the programmable controller and a display screen for displaying drug entries from the drug library. Each of the associated sets of drug delivery parameters includes information selected from a group of parameters including drug concentration, drug delivery rate, drug dose, and bolus size. The electronically loaded drug library contains a list of available mode options specifying the units available for expressing drug delivery information, and the drug infusion pump offers the user the list of available mode options from which to make a selection when the electronically loaded drug library is in the pump. In the case of a syringe pump, the electronically loaded drug library may include a list of names of syringe manufacturers identifying syringes that can be used in the drug infusion pump, and the drug infusion pump offers the user the list of names of syringe manufacturers from which to make a selection when the electronically loaded drug library is in the pump. The loaded drug library may include a list of syringe sizes identifying syringes that can be used in the drug infusion pump, and the drug infusion pump offers the user the list of syringe sizes from which to make a selection when the electronically loaded drug library is in said pump. In the case of a peristaltic pump, the electronically loaded drug library may include a list of infusion set manufacturers. A loaded drug library may include a set of features, each of which is either be toggled on or off, and the pump offers the user only the features from among the set of features that are toggled on when the electronically loaded drug library is in said pump.

FIG. 1B is another view of a portion of the example patient care unit 12 shown in FIG. 1A, according to aspects of the subject technology. FIG. 1B shows two of the fluid infusion pumps 18, 20 mounted at either side of a control unit 14, and the displays and control keys of each, with the programming module being capable of programming both infusion pumps. The infusion device includes a door 5a and a handle 5b that operates to lock the door in a closed position for operation and to unlock and open the door for access to the internal pumping and sensing mechanisms and to load administration sets for the pump. When the door 5a is open, the tube can be connected with the pump 20. When the door 5a is closed, the tube is brought into operating engagement with the pumping mechanism, the upstream and downstream pressure sensors, and the other equipment of the pump. A display 5c, such as an LED display, is located in plain view on the door in this embodiment and may be used to visually communicate various information relevant to the pump 20, such as alert indications (e.g., alarm messages).

Display 5c of each pump module 18, 20 may present a scancode 200 for identifying the module. As will be described further, each scancode may facilitate programming of the pump module (and/or PCU) by way of directly providing configuration information to a scanner based system when the scancode is scanned by a scanner. The system may then process the information and provide programming instructions back to the PCU 12 (or module) via a network transmission channel 31 and network connection 52. Control keys 5e-h exist for programming and controlling operations of the infusion pump as desired including, for example, for activating a presentation of a respective scancode on the display 5c. In some implementations, the control keys may be presented as interactive elements on the display 5c (e.g., touchscreen display). The infusion device and/or infusion pump may also include audio alert equipment in the form of a speaker (not shown).

The control unit 14 of the infusion device 12 includes a display 6a for visually communicating various information, such as the operating parameters of a connected pump and alert indications and alert messages, and control keys 6b and 6c for selecting and/or setting control parameters and/or options for controlling the infusion device 12 and connected modules. The control unit 14 may also include a speaker to provide audible alerts. In some implementations, the display 6a may be implemented as a touchscreen display. In such implementations, the control keys 6b may be omitted or reduced in number by providing corresponding interactive elements via a graphical user interface presented via the display 6a. In some implementations, each control key 6b (or 6c) may select a corresponding option displayed in display 6b.

The control unit 14 may include one or more communications systems (as described with regard to FIG. 1A) with which the control unit 14 may communicate with external equipment such as a medical facility server or other computer and with a portable processor, such as a handheld communication device or a laptop-type of computer, or other information device that a clinician may have to transfer information as well as to download drug libraries to a programming module 16, 18, 20, 22 (such as pump 20). A communication module may be used to transfer access and interaction information for clinicians encountering the programming module or device coupled therewith (e.g., pump 20 or bar code scanner). As described previously, the communications system may include one or more of a radio frequency (RF) system, an optical system such as infrared, a BLUETOOTH™ system, or other wired or wireless system. The bar code scanner and communications system may alternatively be included integrally with the infusion pump 20, such as in cases where a programming module is not used, or in addition to one with the control unit 14. Further, information input devices need not be hard-wired to medical instruments, information may be transferred through a wireless connection as well. Additionally, other types of modules may be connected to the pump modules or to the programming module such as a syringe pump module, patient controlled analgesic module, End Tidal CO₂ monitoring module, oximeter monitoring module, or the like.

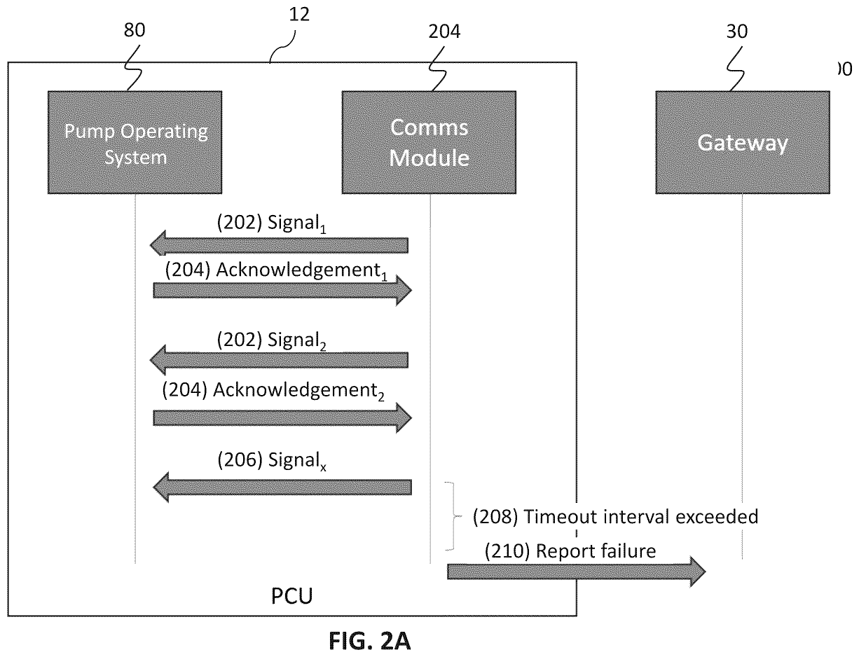

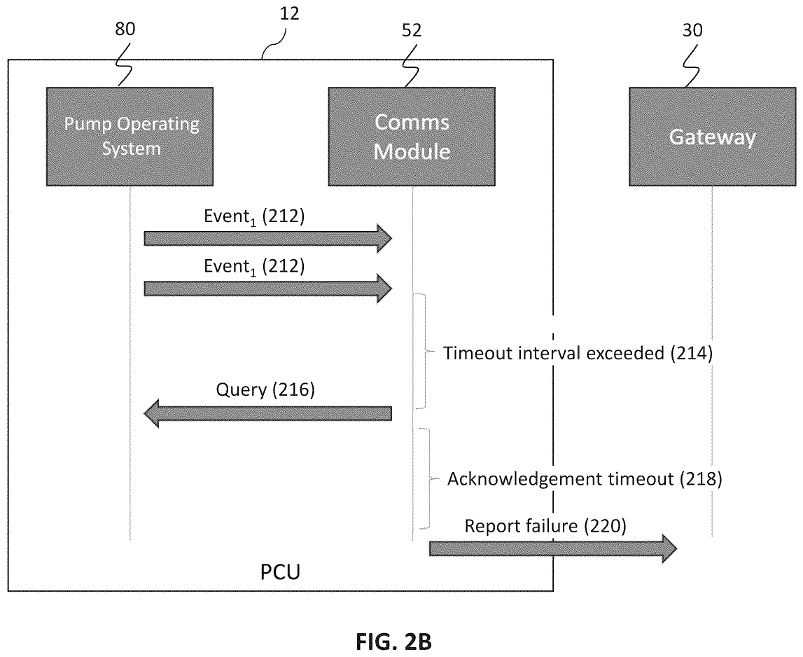

FIG. 2A depicts an example sequence diagram of a first example system for detecting failure of an infusion device, according to various aspects of the subject technology. A communication module 52 monitors a pump operating system 80 at periodic intervals to determine an operational status of the pump operating system 80. As described previously, pump operating system(s) 80 may generally include the hardware and/or software of the PCU 12 and/or one or more of its connected functional modules 16-22 that is responsible for device operation. In this regard, an active pump operating system 80 may include software responsible for pump operations or control circuitry such as CPU 50 (or a respective microprocessor/processor 70) executing software instructions stored in disk 56, RAM 58, 72, ROM 74 or other connected instruction store. Pump operating system 80 may report its health status by way of a processor 50 (and/or a microprocessor 70) transmitting a predetermined signal (which may or may not include other related status information) to communication module 52. In some implementations, communication module 52 may infer operational health by way of detecting traffic from the pump operating system 80 to an external server (which may be brokered by the communication module 52 or network interface 51).

As will be described further, the communication module 52 may periodically (e.g., every 5, 10, 15 seconds, etc.) send a signal (202) to the pump operating system 80 and wait to receive an acknowledgement of the signal (204). The communication module 52 is programmed with a timeout interval and, if the pump operating system 80 fails to acknowledge a signal sent by the communication module 52 within the time period set by the timeout interval then the communication module 52 establishes that the operational status of the pump operating system 80 cannot be determined and may infer an indication of a failure of the pump operating system 80.

The timeout interval may be a system default period of time or dynamically established based on one or more predetermined technical factors associated with the infusion pump or one or more clinical factors associated with a patient receiving a therapy from the infusion pump, the therapy, or a care area associated with the patient. Technical factors may include, for example, the connectivity type (e.g., ethernet, Wi-Fi, cellular, etc.) or quality (e.g., data rate, signal strength, interference, latency) with a local access point on network 10, the number of modules attached to PCU, or a status being reported to the communication module 52 by the PCU (e.g., whether an alert condition is occurring). For example, if the network type or quality are associated with slower transmission, the timeout interval may be shorter than if the transmission is faster to ensure any notification of failure is received in a timely fashion. Clinical factors may include the type or criticality of the care area or the patient, a type of fluid being administered by the PCU (or particular functional module), acuity of the patient, the particular clinician assigned to the care unit or the PCU or patient, and the like.

In the depicted example, the communication module sends a signal (202) at periodic intervals (e.g., every 5 s) and waits for an acknowledgement (204). The pump operating system acknowledges signal₁ and signal₂ within the set time interval (202). The communication module 52 continues to periodically send signals up until signalₓ (206) but the time interval expires (208) before an acknowledgment is received for signalₓ. Accordingly, the communication module 52 cannot determine the status of the pump operating system, infers a failure has occurred, and proceeds to communicate with a remote gateway 30 regarding the failure to determine the operational status of the pump operating system (210).

According to various implementations, the gateway 30 may include a hospital information server or other server configured to monitor medical devices and to message other connected computing devices 32 when a failure is detected. In some implementations, gateway 30 includes an ancillary treatment device such as a multiparameter monitor (MPM) associated with the PCU. As will be described further, when a failure is detected, the communication module 52 may determine one or more reasons for the failure and provide a report to the gateway 30. According to some implementations, the report may include pump information, clinician ID (who logged in last), patient ID, alarm status, pump status (paused, stopped, infusing), or other info available to the pump/PCU.

FIG. 2B depicts an example sequence diagram of a second example system for detecting failure of an infusion device, according to various aspects of the subject technology. In the example of FIG. 2B, the infusion events are used as a proxy for the operational status of the pump operating system(s) 80. Infusion events may include, for example, a status of an active infusion, which may include operational parameters and infusion data such as flow rate, volume infused, measured physiological parameters (e.g., heart rate, oxygen, etc.), as well as order identifier, patient identifier, pump identifier, and other related information to identify the infusion. The infusion events may be, for example, handled or detected by the communication module 52 when they are provided to the gateway for storage at an EMR (electronic medical record) server, or for storage in connection with care of the patient (e.g., in the patient's EMR).

Each infusion event provided by the operating system 80 (212) and received/detected by the communication module 52 functions as a status message that satisfies the module's monitoring criteria. The timeout interval is reset after each event message is detected. If the timeout interval is then exceeded (214) before receiving/detecting another status message then the communication module 80 queries (216) the pump operating system 80 for a specific health signal to confirm that the pump operating system 80 is still functioning. A predetermined acknowledgement timeout period is set based on the same or similar criteria as the previously described timeout interval. If the communication module 52 does not receive the health signal in response to the query within the acknowledgement timeout period (218) then a failure report is transmitted (220) to the gateway 30.

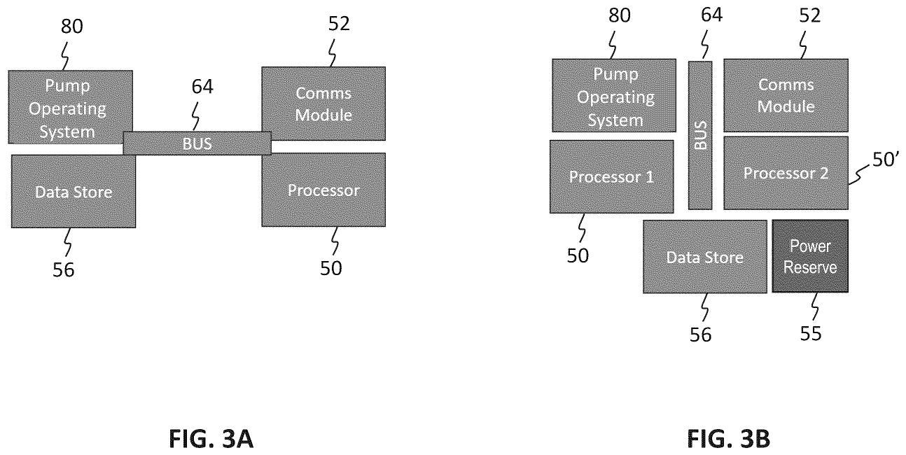

FIGS. 3A and 3B depict example architectural arrangements of the communication module 52 with respect to the pump operating system, according to various aspects of the subject technology. In FIG. 3A, the communication module 52 and the pump operating system 80 share a single processor 50 (or single processor group). In the depicted example, the communication module 52 and the pump operating system 80 are separate processes, virtual machines or containers (e.g., Kubernetes). Messages are exchanged via a container manager over a common bus 64, and/or indirectly via a common data store 56.

In FIG. 3B, the communication module 52 and the pump operating system 80 are separate and independent hardware, each controlled by a separate processor 50, 50′. Messages are exchanged via a container manager over a common bus 64, and/or indirectly via a common data store 56. In some implementations, the communication module 52 may be connected to a power reserve 55, which can provide power to the communication module 52 in the case of a failure of the PCU 12 and/or pump operating system 80, or other failure of the main power source. In this regard, the communication module 52 may continue to operate, monitor, and communicate with remote system(s) based on power from power reserve 55.

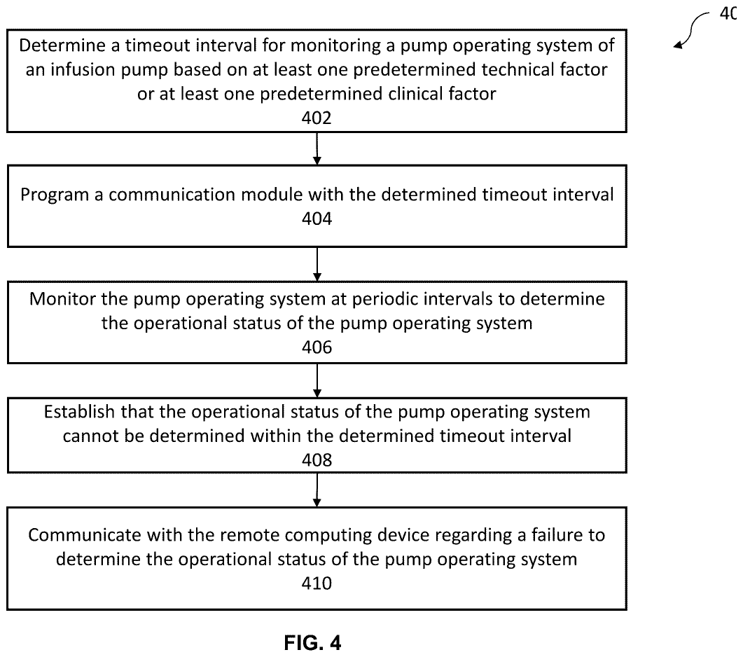

FIG. 4 depicts a first example process flow diagram 400 for detecting failure of an infusion pump, according to aspects of the subject technology. For explanatory purposes, the various blocks of example process 500 are described herein with reference to FIGS. 1-3, and the associated components and/or processes described herein. The one or more of the blocks of process 400 may be implemented, for example, by a PCU 12 or associated module 16, 18, 20, 22 or control unit 14, or processor thereof. In some implementations, one or more of the blocks may be implemented, at least in part, based on one or more machine learning algorithms. In some implementations, one or more of the blocks may be implemented apart from other blocks, and by one or more different processors or devices. Further, for explanatory purposes, to the extent that the blocks of example process 400 are described as occurring in serial, or linearly, in some implementations, multiple blocks of example process 400 may occur in parallel (e.g., blocks 402 and 404 may occur in parallel). In addition, the blocks of example process 400 need not be performed in the order shown and/or one or more of the blocks of example process 400 need not be performed.

A timeout interval for monitoring a pump operating system 80 of an infusion pump is determined based on at least one predetermined technical factor or at least one clinical factor (402). As will be described further, technical factors may include conditions and events associated with the infusion pump, while clinical factors may include conditions and events associated with a patient receiving a therapy from the infusion pump, the therapy, or a care area associated with the patient. The timeout interval may be determined by a processor 50 of the control unit 14 or, in some implementations, by a processor/microprocessor 70 of a functional module. In some implementations, the timeout interval may be determined by a processor 50′ of the communication module 52 itself.

The information utilized to determine the timeout interval may be retrieved from external systems, for example, from an internal data store 56 or an external data store 37 or external server or other operatively connected device (e.g., server 30 or remote terminal 32). In some implementations, the timeout interval may be set by way of user input via a user interface. In some implementations, the information utilized may be derived from operational data, parameters, and/or measurements from integrated systems.

Technical factors may include, for example, how many functional modules are currently attached to a control unit 14, a type or signal strength or round trip time of a network connection (e.g., of a wireless connection), battery information, or a hardware event detected at the device, such as a loading event.

In some implementations, a processor may determine the number of functional modules connected to the control unit. The determination may be by interrogation of the connection ports or based on stored data in memory of the control unit 14 indicating how many devices (and what type) are connected. The processor may then determine the timeout interval based on the number of functional modules. In this regard, the duration of the timeout interval may decrease inversely according to an increase in the number of functional modules. For example, the timeout may be reduced to 2 s from 5 s in response to the number of connected modules increasing from three to four connected modules.

In some implementations, the processor may detect a signal strength of a wireless connection between the communication module and a wireless access point, and determine the timeout interval based on the signal strength. Similar to the previous example, the duration of the timeout interval may decrease inversely according to an increase in the signal strength. In some implementations, the processor may detect that the PCU (or connected module) was switched from a fixed power source (e.g., AC power) to a battery (e.g., DC power) and, in response, increase the timeout interval to a predetermined interval that is greater than the current timeout interval to conserve power. In some implementations, the processor may monitor the capacity of the battery over time and, responsive to the battery reaching a threshold (reduced) capacity, the timeout interval may be reduced to a second predetermined interval that is less than the current predetermined interval. In this regard, the reduced interval may compensate for expected errors that may be produced responsive to reduced power. While the battery capacity may be reduced faster, an alarm may be generated either at the device or sent to a remotely connected device to alert a clinician that the battery capacity is critical and may cause a system failure.

In some implementations, sensors within the PCU or a functional module may detect that an infusion pump is undergoing a loading event (e.g., a syringe is being inserted into a syringe pump or an IV infusion set is being loaded into a pumping mechanism). Responsive to the loading event, the frequency of the monitoring may be increased, and/or the timeout interval reduced, for a predetermined amount of time corresponding to the expected loading time (e.g., 30 seconds).

In some implementations, clinical factors affecting the timeout interval may include the number of clinicians currently staffed in a care area associated with the PCU. The timeout interval may be set to a first threshold interval responsive to the number of clinicians satisfying a first threshold number and may be set to a second threshold interval responsive to the number of clinicians satisfying a second threshold number.

In some implementations, the threshold may be set based on patient acuity. In this regard, the timeout interval may be reduced when the patient associated with the PCU is identified as being in a high-risk category. A high-risk category may be inferred by the assigned care unit. For example, if the patient associated with the infusion pump, or the infusion pump itself, is associated with a high-risk care area (e.g., the ICU or NICU) then the timeout may be adjusted accordingly (e.g., reduced). In some implementation, the timeout interval may be adjusted or set based on the fluid being infused by the infusion pump. For example, controlled substances may be associated with a higher frequency of status messages and/or lower timeout intervals. In this regard, the timeout interval may be reduced when the therapy is a medication identified as a predetermined controlled substance.

In some implementations, the duration of the timeout interval may be reduced in response to the PCU, functional module, or associated component, experiencing an alarm condition. The processor may detect, within a predetermined window of time, one or more alarm conditions associated with a therapy being administered by the device and determine a new timeout interval and increase a frequency of periodic communication messages being sent between the communication module and the pump operating system. For example, the processor may instruct an increase in the frequency of status messages being sent by the pump operating system to the communication module (or through the module to an external server or device). In implementations where the status messages are responsive to signals or queries from the communication module, the communication module may increase the frequency of those signals or queries. The timeout interval may also be reduced accordingly.

According to various implementation, the PCU 14 or a computing device associated with the PCU (e.g., a connected mobile device or terminal 32) may provide a user interface for adjusting how the timeout interval is determined based on the at least one predetermined technical factor or the at least one clinical factor. The interface may include, for example, one or more controls that enable a user to set whether the timeout interval is increased or decreased based on one or more predetermined conditions. For example, a user may set whether the timeout interval is increased or decreased when power is switched to the battery, and/or whether the timeout interval is increased or decreased when the power level of the battery reaches a threshold capacity (e.g., 4 Ah). The same or similar selections may be made for operating parameters (e.g., volume infused, etc.) or sensor readings (e.g., temperature reaches a certain threshold, an occlusion is detected, etc.).

Continuing with the example process 400 of FIG. 4, the communication module 52 is programmed with the determined timeout interval (404). As previously described, the communication module is separate and independent of the pump operating system and is configured to communicate with the pump operating system 80 and to communicate with a remote computing device regarding an operational status of the pump operating system. When the timeout interval is determined by a component external to the communication module 52, that component or a processor associated with the component may program the communication module. In some implementations, the communication module may self-configure based on information being provided to it. For example, timeout interval may be provided to a memory or data store, and the communication module may access the memory or data store to obtain the timeout interval.

The communication module 52 monitors the pump operating system 80 at periodic intervals to determine the operational status of the pump operating system (406). According to various implementations, the monitoring may include the communication module receiving/detecting respective status messages at periodic intervals, indicating the pump operating system is operating correctly. As described with regard to FIG. 2A, the communication module 52 may periodically send the pump operating system a signal (202) and wait to receive an acknowledgement (204) of the signal from the pump operating system. As described with regard to FIG. 2B, the communication module 52 may detect (and/or pass through) infusion events being transmitted from the pump operating system to an external system (e.g., an EMR server) and reset the timeout interval after each infusion event is detected. As part of the monitoring, the communication module 52 determines whether each subsequent infusion event was transmitted within the timeout interval.

The communication module establishes, during the monitoring, that the operational status of the pump operating system 80 cannot be determined within the determined timeout interval (408). According to various implementations, the timeout interval is reset when the pump operating system 80 is determined to be operating properly, for example, by satisfying any threshold conditions for operation, including sending a predetermined signal to the communication module 52 or acknowledging a signal sent to it by the communication module. When the timeout interval expires then the communication module may infer that there is a failure of the pump operating system.

In some implementations, such as described with regard to FIG. 2B, not determining the operational status includes the communication module not detecting a respective infusion event (212) before the timeout interval expires (214). The communication module may then query (216) the pump operating system for a health signal. The communication module may then establish that the status of the pump operating system cannot be determined, or that there is a failure, if the pump operating system does not response to the query with the requested health signal before a predetermined acknowledgement timeout period elapses (218).

Continuing with the example process 400 of FIG. 4, responsive to the communication module 52 not determining the operational status of the pump operating system 80 within the determined timeout interval, the communication module communicates with the remote computing device regarding a failure to determine the operational status of the pump operating system (410). In some implementations, a notification or alert is sent to a remote device, such as the previously described information server 30, a terminal 32 (e.g., at a nurses station), or a mobile device 32 associated with a clinician.

In some implementations, when the communication module 52 is reporting a failure, the communication module 52 may retrieve one or more log entries from a log storing infusion events accumulated during a therapy currently being provided by the infusion pump, and send the log entries to the remote computing device/gateway. For example, the log entries may be sent in a report together with the previously described notification or alert.

Further reference is made to the disclosed timeout intervals, and corresponding clinical and technical factors disclosed herein. According to various implementations, a timeout interval may be based on one or more factors not limited to, for example, the care area associated with the infusion device, number of drugs provided by the infusion device or to the patient, number of modules/active channels providing therapy to the patient, drugs being administered, connectivity type of the communication module (e.g., ethernet vs Wi-Fi), status of pumps, and the like. In some implementations, the timeout interval may be based on the signal strength of a wireless access point. For example, if signal strength is poor it may be harder to inform the server and warrant more scrutiny of the device. A weak transmissions signal (measured by conventional means) between the communication module 52 and the access point (e.g., a local wireless router) or reporting server may result in a shorter timeout.

The number of infusion events occurring at any one time may increase with the number of infusion channels/modules that are operating. Thus, the number of modules may infer a more likelihood of errors in a critical environment, also resulting in a desire for a reduced timeout interval. Battery power may also be monitored and the timeout interval increased to conserve power when the battery is currently the only source of power. Additionally, or in the alternative, as battery level decreases, the timeout may be lengthened so as to not consume power. In some implementations, the timeout interval may be reduced due to chance that the lower power supply may result in more failures.

As described previously, the system may provide a user interface to enable user configuration of how a timeout interval is calculated. For example, for a given condition (e.g., low battery), a mode can be selected for handling the condition. Modes may also be tailored to each care area or other clinical factors. The user interface may allow a user to weigh factors positively or negatively. For example, a user may select a mode to increase the timeout or reduce the timeout for a given condition. For a particular care area or entire fleet at site A the user may select to use inverse power dynamicity where, as power goes down the timeout goes up, or the user may choose to select a linear link (e.g., increased ping and increased report of battery low condition).

Clinical factors considered by the system may include the type of drug used in an infusion, the hydration of the patient, or whether the fluid used is for hydration or critical care. For example, dopamine has half-life of 2 minutes, and thus the clinician is expected to be able to respond more quickly as an interruption to the therapeutic effect of dopamine can be detrimental to the patient. Accordingly, the system may categorize drugs as either high or low risk and set predetermined timeout intervals (or adjustment to timeout intervals) based on the category of drug. In this regard, the system may be configured to alert more aggressively when high risk rugs are used in an infusion (e.g., by increasing the frequency of status checks by the communication module and/or reducing the timeout interval).

Patient acuity (high/low acuity) may be inferred based on care area, and the timeout interval adjusted accordingly. For example, when comparing a high-risk patient (e.g., eggshell patient) to a healthy patient, a predetermined extended timeout interval may be attributed to the healthy patient while a predetermined reduced timeout interval may be attributed to the high-risk patient (e.g., there may be more adverse effects to the eggshell patient if the pump systems fail).

The timeout interval may also be set or adjusted based on how many clinicians are presently on staff in a care area associated with the infusion device. In this regard, the timeout interval may be increased when patients in an understaffed care area are expected to wait longer. However, certain care areas that are understaffed may be configured for a reduced timeout interval (depending on how facility tolerates risk). In some implementations, the system may factor how many resources facility has to respond to a failure report and adjust the timeout interval based on indexing a lookup table based on the number of resources. The specific timeout intervals for these various conditions may be predetermined in a lookup table or set by a user via the previously described user interface.

In some implementations, the communication module 52 may be configured to report status of the pump operating system to multiple servers/gateways. For example, each server or gateway may subscribe to infusion events from the communication module, and/or the communication may register with each server (e.g., that it will be reporting status of the pump operating system). When the communication module detects or infers a failure of the pump operating system, the communication module may then publish a message to all subscribed/registered servers/gateways.

According to various implementations, the disclosed system includes a secondary failure mechanism. As an example, the communication module 52 may communicate an acknowledgement timeout to the server 30 (or device or gateway to which it is reporting operational status of the pump operating system). That is, the communication may report when to expect the next status message (e.g., within the next 15 s), or provide a periodic expectation (e.g., every 15 s), either of which may be different from a current expectation. In some implementations, the server may maintain a default static expectation (e.g., 30 s), which may be then changed based on information received from the communication module 52. In this manner, the server/gateway may further infer a failure if a status report is not received from the communication module 52 by the acknowledgment timeout currently set at the server/gateway.

In some implementations, the communication module 52 may report a status ok message to the server/gateway each time a signal is received from the pump operating system 80. Accordingly, if the pump operating system reports periodically then the communication module 52 will report to the server/gateway periodically. The timeout interval used by the communication module to monitor the pump operating system may also be communicated to the server/gateway so that the server/gateway knows when to expect subsequent status reports from the communication module. If the PCU or communication module adjusts the internal timeout interval, then the same adjustment can be communicated to the server/gateway.

In some implementations, a timeout interval may be determined, set, or adjusted based on a status of the pump and/or prior infusion events. For example, a functional module 16-22 that is alarming a lot, or is in a state of alarming at the time of a status report, may be indicative of a higher risk of technical failure. The frequency of status reports and/or respective timeout interval may be adjusted (e.g., to increase frequency or reduce timeout).

Even small errors at module may be compounded if the pump operating system is failing. Accordingly, the system may be set to be more vigilant in monitoring the pump operating system when errors are occurring. Battery sensor readings outside of expected readings over a period of time (e.g., as measured by a sensor or from a log), or a spike in abnormal measurements, may be indicative of a condition warranting heightened scrutiny. The communication module may then increase frequency of timeouts just in case something happens. As another example, if the infusion device has been fighting occlusion alarms from a module, then the flow continuity from that module could be considered unreliable or suspect. Each incident may only be a small error on its own, however, a history of smaller failures (e.g., reaching a threshold number of alerts over a predetermined window of time) can cause the system to be more vigilant in monitoring, increasing the frequency of monitoring and/or reducing the timeout interval (e.g., by a predetermined amount).

In some implementations, the communication module 52 may monitor a log of events and infer the stability based on how many log events (and in some implementations, type) occurred over the window of time. Additionally, or in the alternative, the PCU or component thereof may monitor sensor readings and increase the monitoring frequency and/or reduce timeout intervals based on the sensor readings being outside of an expected or predetermined range. For example, if temperature measured by a temperature sensor starts to rise beyond a threshold level, then the system may infer that one or more processes are causing the temperature increase and the communication module may begin to monitor more closely.

Some application failures have been known to occur in the presence of a certain physical condition or when a set of events are triggered. For example, pump failures may occur during loading of an infusion set, or when loading a syringe into a syringe pump. Accordingly, the system may be programmed with predetermined conditions in which the pump operating system has a higher chance to experience failure and increase monitoring during those conditions. Accordingly, the system may reduce the timeout interval and/or increase monitoring frequency responsive to one or more sensor being triggered, upon and/or during a loading event, or when increased data or network traffic is detected. The mapping of the conditions to adjustment may be set in a lookup table, and may be further modified based on user input.

In some implementations, the pump operating system may not be able to report an error upon failure, even if queried. Accordingly, in some implementations, responsive to detecting a failure (e.g., when the timeout interval expires), the communication module 52 may monitor/interrogate a log storing infusion events accumulated during a therapy currently being provided by the infusion device, and identify a trend in the logged data. Machine learning may be used to determine whether the logged data in indicative of a cause of failure. The communication module may then retrieve one or more log entries and/or generate a description of the potential cause of the failure, and send the one or more log entries or description to the server/gateway when communicating with the server/gateway regarding the failure.

Many of the above-described example process 500 and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

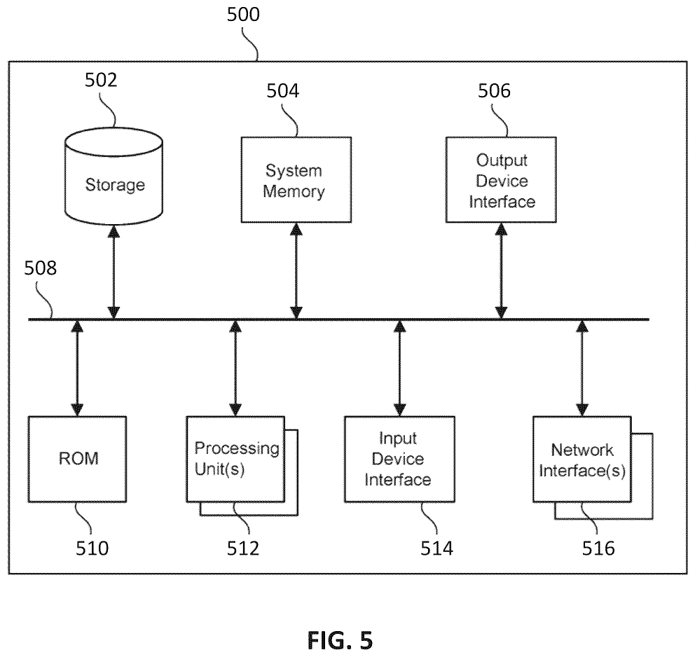

FIG. 5 is a conceptual diagram illustrating an example electronic system 500 for detecting failure of an infusion pump, according to aspects of the subject technology. Electronic system 500 may be a computing device for execution of software associated with one or more portions or steps of processes 300, 350, 400, and 500 or components and methods provided by FIGS. 1-5, including but not limited to computing hardware within servers 30, 34, terminal 32, 206, medical device 12, and/or any computing devices or associated modules or terminals disclosed herein. Electronic system 500 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 500 may include various types of computer readable media and interfaces for various other types of computer readable media. In the depicted example, electronic system 500 includes a bus 508, processing unit(s) 512, a system memory 504, a read-only memory (ROM) 510, a permanent storage device 502, an input device interface 514, an output device interface 506, and one or more network interfaces 516. In some implementations, electronic system 500 may include or be integrated with other computing devices or circuitry for operation of the various components and methods previously described.

Bus 508 collectively represents all system, peripheral, and chipset buses that communicatively connect the numerous internal devices of electronic system 500. For instance, bus 508 communicatively connects processing unit(s) 512 with ROM 510, system memory 504, and permanent storage device 502.

From these various memory units, processing unit(s) 512 retrieves instructions to execute and data to process, in order to execute the processes of the subject disclosure. The processing unit(s) can be a single processor or a multi-core processor in different implementations.

ROM 510 stores static data and instructions that are needed by processing unit(s) 512 and other modules of the electronic system. Permanent storage device 502, on the other hand, is a read-and-write memory device. This device is a non-volatile memory unit that stores instructions and data even when electronic system 500 is off. Some implementations of the subject disclosure use a mass-storage device (such as a magnetic or optical disk and its corresponding disk drive) as permanent storage device 502.

Other implementations use a removable storage device (such as a floppy disk, flash drive, and its corresponding disk drive) as permanent storage device 502. Like permanent storage device 502, system memory 504 is a read-and-write memory device. However, unlike storage device 502, system memory 504 is a volatile read-and-write memory, such as, random access memory. System memory 504 stores some of the instructions and data that the processor needs at runtime. In some implementations, the processes of the subject disclosure are stored in system memory 504, permanent storage device 502, and/or ROM 510. From these various memory units, processing unit(s) 512 retrieves instructions to execute and data to process in order to execute the processes of some implementations.

Bus 408 also connects to input and output device interfaces 514 and 506. Input device interface 514 enables the user to communicate information and select commands to the electronic system. Input devices used with input device interface 514 include, e.g., alphanumeric keyboards and pointing devices (also called “cursor control devices”). Output device interfaces 506 enables, e.g., the display of images generated by the electronic system 500. Output devices used with output device interface 506 include, e.g., printers and display devices, such as cathode ray tubes (CRT) or liquid crystal displays (LCD). Some implementations include devices such as a touchscreen that functions as both input and output devices.

Also, as shown in FIG. 5, bus 508 also couples electronic system 500 to a network (not shown) through network interfaces 516. Network interfaces 516 may include, e.g., a wireless access point (e.g., Bluetooth or WiFi) or radio circuitry for connecting to a wireless access point. Network interfaces 516 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 500 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology As Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification

Clause 1. A computer-implemented method for detecting failure of an infusion pump, comprising: determining a timeout interval for monitoring a pump operating system of an infusion pump based on at least one predetermined technical factor associated with the infusion pump or at least one clinical factor associated with a patient receiving a therapy from the infusion pump, the therapy, or a care area associated with the patient; programming a communication module with the determined timeout interval, the communication module being separate and independent of the pump operating system and configured to communicate with the pump operating system and to communicate with a remote computing device regarding an operational status of the pump operating system; monitoring, by the communication module, the pump operating system at periodic intervals to determine the operational status of the pump operating system; establishing, by the communication module, during the monitoring, that the operational status of the pump operating system cannot be determined within the determined timeout interval; and responsive to the communication module not determining the operational status of the pump operating system within the determined timeout interval, by the communication module, communicating with the remote computing device regarding a failure to determine the operational status of the pump operating system.

Clause 2. The computer-implemented method of Clause 1, wherein monitoring the pump operating system comprises receiving, by the communication module from the pump operating system, respective status messages at periodic intervals, indicating the pump operating system is operating correctly; and wherein the communication module not determining the operational status of the pump operating system within the determined timeout interval comprises the communication module not receiving a status message from the pump operating system within the determined timeout interval.

Clause 3. The computer-implemented method of Clause 1, wherein the monitoring comprises: detecting infusion events being transmitted from the pump operating system; resetting the timeout interval after each infusion event is detected; and determining whether each subsequent infusion event is detected before the timeout interval expires, wherein the communication module not determining the operational status of the pump operating system within the determined timeout interval comprises: the communication module not detecting a respective infusion event before the timeout interval expires, the communication module querying the pump operating system for a health signal responsive to not detecting the respective infusion event from the pump operating system, and the communication module not receiving the health signal from the pump operating system within a predetermined acknowledgement period.

Clause 4. The computer-implemented method of any one of Clauses 1-3, wherein the infusion pump is removably connected to a control unit, the control unit being configured to removably connect to and control a plurality of functional modules, including the infusion pump, the method further comprising: determining a number of functional modules connected to the control unit; and determining the timeout interval based on the number of functional modules, wherein a duration of the timeout interval decreases inversely according to an increase in the number of functional modules.

Clause 5. The computer-implemented method of any one of Clauses 1-4, further comprising: detecting a signal strength of a wireless connection between the communication module and a wireless access point; and determining the timeout interval based on the signal strength, wherein a duration of the timeout interval decreases inversely according to an increase in the signal strength.

Clause 6. The computer-implemented method of any one of Clauses 1-5, further comprising: detecting that the infusion pump was switched to being powered by a battery; and increasing, responsive to the infusion pump being powered by the battery, the timeout interval to a first predetermined interval greater than the determined timeout interval.

Clause 7. The computer-implemented method of Clause 6, further comprising: detecting that a capacity of the battery reached a threshold capacity; and decreasing, responsive to battery reaching the threshold capacity, the timeout interval to a second predetermined interval that is less than the determined timeout interval and the first predetermined interval.

Clause 8. The computer-implemented method of any one of Clauses 1-7, further comprising: providing a user interface comprising one or more controls for adjusting how the timeout interval is determined based on the at least one predetermined technical factor or the at least one clinical factor, wherein the one or more controls enables a user to set whether the timeout interval is increased or decreased based on one or more predetermined conditions.

Clause 9. The computer-implemented method of any one of Clauses 1-8, further comprising: periodically sending, by the communication module, communication messages to the remote computing device regarding the operational status of the pump operating system; and sending at least one communication message together with an indication of a subsequent time when the remote computing device should expect a subsequent communication message, wherein if the subsequent communication message is not received by the remote computing device by the subsequent time then the remote computing device infers that a failure has occurred at the communication module.

Clause 10. The computer-implemented method of any one of Clauses 1-9, further comprising: receiving an indication that the infusion pump is undergoing a loading event; and responsive to the loading event, increasing a frequency of the monitoring for a predetermined expected loading time.

Clause 11. The computer-implemented method of any one of Clauses 1-10, further comprising: determining the timeout interval based on a number of clinicians currently staffed in a care area associated with the infusion pump, wherein the timeout interval is set to a first threshold interval responsive to the number of clinicians satisfying a first threshold number and is set to a second threshold interval responsive to the number of clinicians satisfying a second threshold number; or a patient acuity of a patient receiving a therapy from the infusion pump, wherein the timeout interval is reduced when the patient is identified as being in a predetermined high risk category; or the therapy provided by the infusion pump, wherein the timeout interval is reduced when the therapy is a medication identified as a predetermined controlled substance.

Clause 12. The computer-implemented method of any one of Clauses 1-12, further comprising: detecting, within a predetermined window of time, one or more alarm conditions associated with a therapy being administered by the infusion pump; and responsive to detecting the one or more alarm conditions within the predetermined window of time, reducing a duration of the determined timeout interval, or increasing a frequency of periodic communication messages being sent from the communication module to the remote computing device regarding an operational status of the pump operating system, wherein if a respective periodic communication message is not received by the remote computing device by a predetermined period of time then the remote computing device infers that a failure has occurred at the communication module.

Clause 13. The computer-implemented method of any one of Clauses 1-13, further comprising, responsive to failing to determine the operational status of the pump operating system for the determined timeout interval: retrieving one or more log entries from a log storing infusion events accumulated during a therapy currently being provided by the infusion pump; and sending the one or more log entries to the remote computing device when communicating with the remote computing device regarding the failure to determine the operational status of the pump operating system.

Clause 14. A infusion system, comprising: an infusion pump; a housing comprising a control circuitry, a non-transitory machine readable medium, and a communication module that is separate and independent of the control circuitry and the infusion pump, wherein the control circuitry is configured to execute a pump operating system stored at least in part on the machine readable medium and to operate the infusion pump according to a set of operating parameters provided to the executed pump operating system, and to provide infusion information pertaining to an infusion to an external server via the communication module, and wherein the communication module is configured to monitor an operational status of the pump operating system and to communicate with a remote computing device regarding the operational status of the pump operating system; wherein the infusion system is configured to: determine a timeout interval for monitoring the pump operating system based on at least one predetermined technical factor associated with the infusion pump or at least one clinical factors associated with a patient receiving a therapy from the infusion pump, the therapy, or a care area associated with the patient; program the communication module with the determined timeout interval; monitor, by the communication module, the pump operating system at periodic intervals to determine the operational status of the pump operating system; establish, by the communication module, during the monitoring, that the operational status of the pump operating system cannot be determined within the determined timeout interval; and responsive to the communication module not determining the operational status of the pump operating system within the determined timeout interval, by the communication module, communicate with the remote computing device regarding a failure to determine the operational status of the pump operating system.

Clause 15. The infusion system of Clause 13, wherein monitoring the pump operating system comprises receiving, by the communication module from the pump operating system, respective status messages at periodic intervals, indicating the pump operating system is operating correctly; and wherein the communication module not determining the operational status of the pump operating system within the determined timeout interval comprises the communication module not receiving a status message from the pump operating system within the determined timeout interval.

Clause 16. The infusion system of Clause 15, wherein the communication module not determining the operational status of the pump operating system within the determined timeout interval further comprises: querying the pump operating system for a health signal responsive to not receiving the status message from the pump operating system within the determined timeout interval and not receiving the health signal from the pump operating system within a predetermined acknowledgement period.

Clause 17. The infusion system of any one of Clauses 14-16, being further configured to: periodically send, by the communication module, communication messages to the remote computing device regarding the operational status of the pump operating system; and send at least one communication message together with an indication of a subsequent time when the remote computing device should expect a subsequent communication message, wherein if the subsequent communication message is not received by the remote computing device by the subsequent time then the remote computing device infers that a failure has occurred at the communication module.

Clause 18. The infusion system of Clause 14, being further configured to: receive an indication that the infusion pump is undergoing a loading event; and responsive to the loading event, increase a frequency of the monitoring for a predetermined expected loading time.

Clause 19. The infusion system of any one of Clauses 14-18, being further configured to: determine the timeout interval based on a number of clinicians currently staffed in a care area associated with the infusion pump, wherein the timeout interval is set to a first threshold interval responsive to the number of clinicians satisfying a first threshold number and is set to a second threshold interval responsive to the number of clinicians satisfying a second threshold number; or determine the timeout interval based on a patient acuity of a patient receiving a therapy from the infusion pump, wherein the timeout interval is reduced when the patient is identified as being in a predetermined high risk category; or determine the timeout interval based on the therapy provided by the infusion pump, wherein the timeout interval is reduced when the therapy is a medication identified as a predetermined controlled substance.

Clause 20. The infusion system of any one of Clauses 14-20, being further configured to: detect, within a predetermined window of time, one or more alarm conditions associated with a therapy being administered by the infusion pump; and responsive to detecting the one or more alarm conditions within the predetermined window of time, reduce a duration of the determined timeout interval, or increase a frequency of periodic communication messages being sent from the communication module to the remote computing device regarding an operational status of the pump operating system, wherein if a respective periodic communication message is not received by the remote computing device by a predetermined period of time then the remote computing device infers that a failure has occurred at the communication module.

Clause 21. The infusion system of any one of Clauses 14-20, being further configured to: provide a user interface comprising one or more controls for adjusting how the timeout interval is determined based on the at least one predetermined technical factor or the at least one clinical factor, wherein the one or more controls enables a user to set whether the timeout interval is increased or decreased based on one or more predetermined conditions.

Clause 22. A communication module for monitoring for failure of an infusion pump, the communication module being separate and independent of control circuity responsible for operating the infusion pump according to a set of operating parameters provided to a pump operating system of the infusion pump, the communication module being configured to: communicate with the pump operating system and with a remote computing device regarding an operational status of the pump operating system; determine a timeout interval for monitoring a pump operating system of an infusion pump based on at least one predetermined technical factor associated with the infusion pump or at least one clinical factors associated with a patient receiving a therapy from the infusion pump, the therapy, or a care area associated with the patient; program a communication module with the determined timeout interval, the communication module being separate and independent of the pump operating system and configured to communicate with the pump operating system and to communicate with a remote computing device regarding an operational status of the pump operating system; monitor, by the communication module, the pump operating system at periodic intervals to determine the operational status of the pump operating system; establish, by the communication module, during the monitoring, that the operational status of the pump operating system cannot be determined within the determined timeout interval; and responsive to the communication module not determining the operational status of the pump operating system within the determined timeout interval, by the communication module, communicate with the remote computing device regarding a failure to determine the operational status of the pump operating system.

Clause 23. The communication module of Clause 22, wherein monitoring the pump operating system comprises sending the pump operating system a signal and waiting to receive an acknowledgement of the signal from the pump operating system; and wherein the communication module not determining the operational status of the pump operating system within the determined timeout interval comprises the communication module not receiving the acknowledgement within the determined timeout interval.

### Further Consideration

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation, or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, . NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information for the message. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. A computer-implemented method for detecting failure of an infusion pump, comprising:
determining a timeout interval for monitoring a pump operating system of an infusion pump based on at least one predetermined technical factor associated with the infusion pump or at least one clinical factor associated with a patient receiving a therapy from the infusion pump, the therapy, or a care area associated with the patient;
programming a communication module with the determined timeout interval, the communication module being separate and independent of the pump operating system and configured to communicate with the pump operating system and to communicate with a remote computing device regarding an operational status of the pump operating system;
monitoring, by the communication module, the pump operating system at periodic intervals to determine the operational status of the pump operating system;
establishing, by the communication module, during the monitoring, that the operational status of the pump operating system cannot be determined within the determined timeout interval; and
responsive to the communication module not determining the operational status of the pump operating system within the determined timeout interval, by the communication module, communicating with the remote computing device regarding a failure to determine the operational status of the pump operating system.

2. The computer-implemented method of claim 1,
wherein monitoring the pump operating system comprises receiving, by the communication module from the pump operating system, respective status messages at periodic intervals, indicating the pump operating system is operating correctly; and
wherein the communication module not determining the operational status of the pump operating system within the determined timeout interval comprises the communication module not receiving a status message from the pump operating system within the determined timeout interval.

3. The computer-implemented method of claim 1, wherein the monitoring comprises:
detecting infusion events being transmitted from the pump operating system;
resetting the timeout interval after each infusion event is detected; and
determining whether each subsequent infusion event is detected before the timeout interval expires,
wherein the communication module not determining the operational status of the pump operating system within the determined timeout interval comprises:
the communication module not detecting a respective infusion event before the timeout interval expires,
the communication module querying the pump operating system for a health signal responsive to not detecting the respective infusion event from the pump operating system, and
the communication module not receiving the health signal from the pump operating system within a predetermined acknowledgement period.

4. The computer-implemented method of claim 1, wherein the infusion pump is removably connected to a control unit, the control unit being configured to removably connect to and control a plurality of functional modules, including the infusion pump, the method further comprising:
determining a number of functional modules connected to the control unit; and
determining the timeout interval based on the number of functional modules, wherein a duration of the timeout interval decreases inversely according to an increase in the number of functional modules.

5. The computer-implemented method of claim 1, further comprising:
detecting a signal strength of a wireless connection between the communication module and a wireless access point; and
determining the timeout interval based on the signal strength, wherein a duration of the timeout interval decreases inversely according to an increase in the signal strength.

6. The computer-implemented method of claim 1, further comprising:
detecting that the infusion pump was switched to being powered by a battery; and
increasing, responsive to the infusion pump being powered by the battery, the timeout interval to a first predetermined interval greater than the determined timeout interval.

7. The computer-implemented method of claim 6, further comprising:
detecting that a capacity of the battery reached a threshold capacity; and
decreasing, responsive to battery reaching the threshold capacity, the timeout interval to a second predetermined interval that is less than the determined timeout interval and the first predetermined interval.

8. The computer-implemented method of claim 1, further comprising:
providing a user interface comprising one or more controls for adjusting how the timeout interval is determined based on the at least one predetermined technical factor or the at least one clinical factor, wherein the one or more controls enables a user to set whether the timeout interval is increased or decreased based on one or more predetermined conditions.

9. The computer-implemented method of claim 1, further comprising:
periodically sending, by the communication module, communication messages to the remote computing device regarding the operational status of the pump operating system; and
sending at least one communication message together with an indication of a subsequent time when the remote computing device should expect a subsequent communication message, wherein if the subsequent communication message is not received by the remote computing device by the subsequent time then the remote computing device infers that a failure has occurred at the communication module.

10. The computer-implemented method of claim 1, further comprising:
receiving an indication that the infusion pump is undergoing a loading event; and
responsive to the loading event, increasing a frequency of the monitoring for a predetermined expected loading time.

11. The computer-implemented method of claim 1, further comprising:
determining the timeout interval based on a number of clinicians currently staffed in a care area associated with the infusion pump, wherein the timeout interval is set to a first threshold interval responsive to the number of clinicians satisfying a first threshold number and is set to a second threshold interval responsive to the number of clinicians satisfying a second threshold number; or
a patient acuity of a patient receiving a therapy from the infusion pump, wherein the timeout interval is reduced when the patient is identified as being in a predetermined high risk category; or
the therapy provided by the infusion pump, wherein the timeout interval is reduced when the therapy is a medication identified as a predetermined controlled substance.

12. The computer-implemented method of claim 1, further comprising:
detecting, within a predetermined window of time, one or more alarm conditions associated with a therapy being administered by the infusion pump; and
responsive to detecting the one or more alarm conditions within the predetermined window of time,
reducing a duration of the determined timeout interval, or
increasing a frequency of periodic communication messages being sent from the communication module to the remote computing device regarding an operational status of the pump operating system, wherein if a respective periodic communication message is not received by the remote computing device by a predetermined period of time then the remote computing device infers that a failure has occurred at the communication module.

13. The computer-implemented method of claim 1, further comprising, responsive to failing to determine the operational status of the pump operating system for the determined timeout interval:
retrieving one or more log entries from a log storing infusion events accumulated during a therapy currently being provided by the infusion pump; and
sending the one or more log entries to the remote computing device when communicating with the remote computing device regarding the failure to determine the operational status of the pump operating system.

14. An infusion system, comprising:
an infusion pump;
a housing comprising a control circuitry, a non-transitory machine readable medium, and a communication module that is separate and independent of the control circuitry and the infusion pump,
wherein the control circuitry is configured to execute a pump operating system stored at least in part on the machine readable medium and to operate the infusion pump according to a set of operating parameters provided to the executed pump operating system, and to provide infusion information pertaining to an infusion to an external server via the communication module, and
wherein the communication module is configured to monitor an operational status of the pump operating system and to communicate with a remote computing device regarding the operational status of the pump operating system;
wherein the infusion system is configured to:
determine a timeout interval for monitoring the pump operating system based on at least one predetermined technical factor associated with the infusion pump or at least one clinical factors associated with a patient receiving a therapy from the infusion pump, the therapy, or a care area associated with the patient;
program the communication module with the determined timeout interval;
monitor, by the communication module, the pump operating system at periodic intervals to determine the operational status of the pump operating system;
establish, by the communication module, during the monitoring, that the operational status of the pump operating system cannot be determined within the determined timeout interval; and
responsive to the communication module not determining the operational status of the pump operating system within the determined timeout interval, by the communication module, communicate with the remote computing device regarding a failure to determine the operational status of the pump operating system.

15. The infusion system of claim 14,
wherein monitoring the pump operating system comprises receiving, by the communication module from the pump operating system, respective status messages at periodic intervals, indicating the pump operating system is operating correctly; and
wherein the communication module not determining the operational status of the pump operating system within the determined timeout interval comprises the communication module not receiving a status message from the pump operating system within the determined timeout interval.

16. The infusion system of claim 15, wherein the communication module not determining the operational status of the pump operating system within the determined timeout interval further comprises:
querying the pump operating system for a health signal responsive to not receiving the status message from the pump operating system within the determined timeout interval and not receiving the health signal from the pump operating system within a predetermined acknowledgement period.

17. The infusion system of claim 14, being further configured to:
periodically send, by the communication module, communication messages to the remote computing device regarding the operational status of the pump operating system; and
send at least one communication message together with an indication of a subsequent time when the remote computing device should expect a subsequent communication message, wherein if the subsequent communication message is not received by the remote computing device by the subsequent time then the remote computing device infers that a failure has occurred at the communication module.

18. The infusion system of claim 14, being further configured to:
receive an indication that the infusion pump is undergoing a loading event; and
responsive to the loading event, increase a frequency of the monitoring for a predetermined expected loading time.

19. The infusion system of claim 14, being further configured to:
determine the timeout interval based on a number of clinicians currently staffed in a care area associated with the infusion pump, wherein the timeout interval is set to a first threshold interval responsive to the number of clinicians satisfying a first threshold number and is set to a second threshold interval responsive to the number of clinicians satisfying a second threshold number; or
determine the timeout interval based on a patient acuity of a patient receiving a therapy from the infusion pump, wherein the timeout interval is reduced when the patient is identified as being in a predetermined high risk category; or
determine the timeout interval based on the therapy provided by the infusion pump, wherein the timeout interval is reduced when the therapy is a medication identified as a predetermined controlled substance.

20. The infusion system of claim 14, being further configured to:
detect, within a predetermined window of time, one or more alarm conditions associated with a therapy being administered by the infusion pump; and
responsive to detecting the one or more alarm conditions within the predetermined window of time,
reduce a duration of the determined timeout interval, or
increase a frequency of periodic communication messages being sent from the communication module to the remote computing device regarding an operational status of the pump operating system, wherein if a respective periodic communication message is not received by the remote computing device by a predetermined period of time then the remote computing device infers that a failure has occurred at the communication module.

21. A communication module for monitoring for failure of an infusion pump, the communication module being separate and independent of control circuity responsible for operating the infusion pump according to a set of operating parameters provided to a pump operating system of the infusion pump, the communication module being configured to:
communicate with the pump operating system and with a remote computing device regarding an operational status of the pump operating system;
determine a timeout interval for monitoring a pump operating system of an infusion pump based on at least one predetermined technical factor associated with the infusion pump or at least one clinical factors associated with a patient receiving a therapy from the infusion pump, the therapy, or a care area associated with the patient;
program a communication module with the determined timeout interval, the communication module being separate and independent of the pump operating system and configured to communicate with the pump operating system and to communicate with a remote computing device regarding an operational status of the pump operating system;
monitor, by the communication module, the pump operating system at periodic intervals to determine the operational status of the pump operating system;
establish, by the communication module, during the monitoring, that the operational status of the pump operating system cannot be determined within the determined timeout interval; and
responsive to the communication module not determining the operational status of the pump operating system within the determined timeout interval, by the communication module, communicate with the remote computing device regarding a failure to determine the operational status of the pump operating system.

22. The communication module of claim 21,
wherein monitoring the pump operating system comprises sending the pump operating system a signal and waiting to receive an acknowledgement of the signal from the pump operating system; and
wherein the communication module not determining the operational status of the pump operating system within the determined timeout interval comprises the communication module not receiving the acknowledgement within the determined timeout interval.
